(19) United States
(12) Patent Application Publication
(10) Pub. No.: US 2017/0112371 A1
(43) Pub. Date: Apr. 27, 2017

(54) ANOSCOPE

(71) Applicant: GEORGE PERCY McGOWN, Brooklyn, NY (US)

(72) Inventor: GEORGE PERCY McGOWN, Brooklyn, NY (US)

(21) Appl. No.: 14/923,662

(22) Filed: Oct. 27, 2015

(51) Int. Cl. A61B 1/31

(52) U.S. Cl. CPC A61B 1/31

(57) ABSTRACT

An anoscope has a tubular body member and a sliding insert that removably covers an opening or window in the body member. The hollow body member is provided on an inwardly facing surface, along a channel or lumen of the body member, with a non-reflective or light-absorbing coating.

### FIELD OF THE INVENTION

This invention relates to an anal port device exemplarily utilizable in the treatment of hemorrhoids.

### BACKGROUND OF THE INVENTION

An anoscope is a device for providing access to the anal canal and lower rectum. It is generally a tubular device that is inserted into the anus and dilates the anus to provide an access passage. In some instances, the anoscope provides a passage for a suturing device to apply purse string sutures to the desired tissue. One common application is for hemorrhoid surgery where the hemorrhoid or adjacent tissue is accessed through the anoscope and purse stringed for subsequent clamping in a circular stapling device which resects and staples the tissue.

Hemorrhoidal disease is a very common condition, affecting more than half of people at age 50. Approximately 500,000 patients receive one or another type of interventional treatment annually in the United States for symptomatic hemorrhoids. Approximately 160,000 patients a year in the U.S. undergo surgical excision of hemorrhoids.

The term “hemorrhoid” is generally used to refer to the disturbing perianal symptoms related to vascular complexes in the lower rectum and anus. This is usually associated with enlargement of this naturally occurring vascular tissue, which is responsible for its subsequent bleeding, prolapsing, thrombosis, itching, burning, etcetera. The word “hemorrhoids” originates from Greek “haimorrhoos” (haimo—hemo+rhein—to flow), which means “flowing with blood.” The word “pile” is a synonym for hemorrhoid, which originates from Latin “pila”—“ball.”

Repetitive straining due to constipation appears to be a leading factor in forming and progressing of hemorrhoids. The chances of having symptomatic hemorrhoids increase with age, pregnancy, obesity, sedimentary life, heavy lifting and genetic predisposition.

Hemorrhoids, located in the rectum, are called internal. Internal hemorrhoids are located within the submucosal layer. External hemorrhoids are located in the anus. Internal and external hemorrhoids have generally different clinical presentation and complications.

Rubber band ligation is the most popular method of treatment of hemorrhoids in the United States. The technique was described by Blaisdell in 1963. It is quick and not expensive. In this procedure, some hemorrhoidal tissue is pulled into the ligator and a rubber band is placed around the base of the pulled tissue. This causes essentially a strangulation of the blood supply to a portion of the internal hemorrhoid and its overlying rectal mucosa. An ischemic necrosis and autoamputation of the hemorrhoid follows in a few days, leaving an open rectal wound, which heals over several days. Significant postprocedural pain, affecting daily routine, is rare and is probably related to the placement of the band too close to the dentate line (pain-sensitive area). Rubber band ligation is very effective for immediate bleeding control of small internal hemorrhoids. Several treatments of a single larger hemorrhoid may be required in order to achieve substantial size reduction.

Anoscopes are typically used to facilitate visual and instrumentation access to hemorrhoids. Anoscopes are basically tubular members which are open at a proximal end (relative to the user) and formed with an opening either at a distal end or in a sidewall. Anoscopes are typically made of metal or metal alloy.

### OBJECTS OF THE INVENTION

It is an object of the present invention to provide an improved anoscope.

A more particular object of the present invention is to provide an anoscope with improved visualization.

These and other objects of the present invention will be apparent from the drawings and descriptions herein. Although every object of the invention is believed to be attained in at least one embodiment of the invention, there is not necessarily any single embodiment that attains all of the objects of the invention.

### SUMMARY OF THE INVENTION

An anoscope, exemplarily utilizable in hemorrhoidal surgery, comprises, in accordance with the present invention, a hollow body defining a longitudinal channel, the hollow body being at least partially open at a proximal end and having an opening or window spaced from the opening at the proximal end. An insert member is movably mounted to the hollow body to cover or block the opening or window during a positioning of the anoscope in an anal canal, the insert member being removable from the channel to uncover the opening or window and permit access to the hemorrhoidal tissues via the hollow body. The hollow body is provided on an inner surface, facing the longitudinal channel with a coating or layer of a non-reflective or light-absorbing material.

The coating or layer may be made of a black polymeric material.

The coating or layer is preferably coextensive with the channel, that is, the entire inner surface of the tubular body member is coated with the non-reflective material.

An anoscope in accordance with the invention provides the user with enhanced visualization as reflections are reduced, if not eliminated. Typically such reflections result from light sources used by the surgeon or medical specialist in order to see anal tissues.

An anoscope pursuant to the present invention highlights the target tissues by removing extraneous, distracting and confusing sensory input.

### BRIEF DESCRIPTION OF THE DRAWINGS

FIG. 1 is a schematic perspective view, partially broken away, of an anoscope assembly in accordance with the present invention.

FIG. 2A is a schematic cross-sectional view of the anoscope assembly of FIG. 1 inserted into an anal canal, showing a first step of a method utilizing the anoscope of FIG. 1.

FIG. 2B is a schematic cross-sectional view similar to FIG. 2A, showing a subsequent step of a method utilizing the anoscope assembly of FIG. 1.

FIG. 3 is a schematic perspective view of another anoscope assembly in accordance with the present invention.

FIG. 4 is a schematic exploded side elevation view of the anoscope assembly of FIG. 3, partially broken away to show a light-absorbing coating or layer in accordance with the present invention.

### DETAILED DESCRIPTION

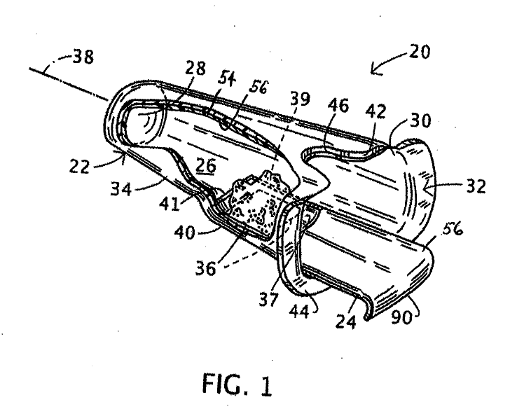

As illustrated in FIG. 1, an anoscope assembly 20 for hemorrhoidal surgery comprises a hollow body 22 and a shutter member 24. Hollow body 22 defines a longitudinal channel or lumen 26 that is closed at a distal end 28 and formed with an opening 30 at a proximal end 32. Opening 30 enables visual inspection of a surgical site and the insertion of instrumentation. Hollow body 22 has a sidewall 34 provided with a rectangular window 36 spaced from distal end 28 and preferably also from distal end 28 of hollow body 22.

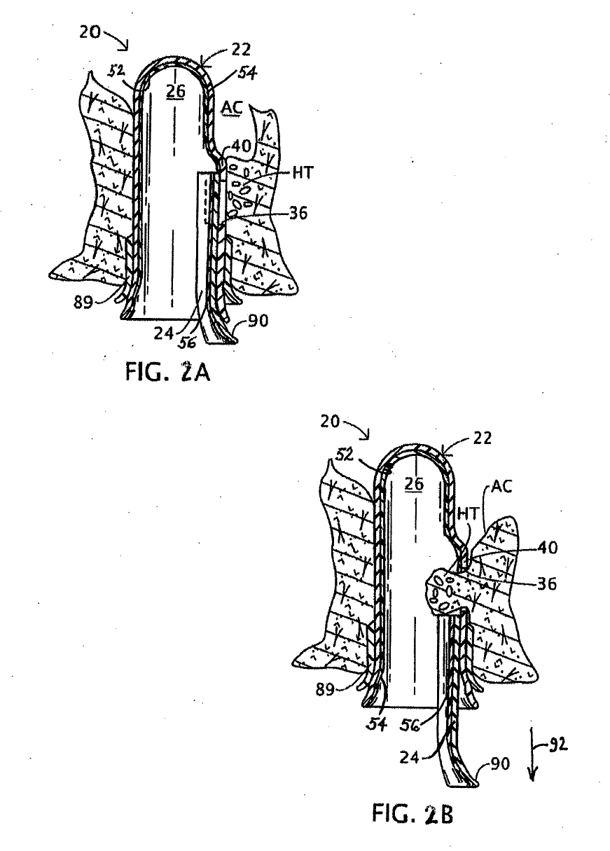

A shutter member or insert 24 is movably mounted to hollow body 22 to cover window 36 during a positioning of anoscope 20 in an anal canal AC, as shown in FIG. 2A. Shutter member 24 is removable from window 36 to permit hemorrhoidal tissues HT to protrude through window 36 into anoscope channel 26, as depicted in FIG. 2B. More specifically, shutter member 24 is slidably mounted to hollow body 22, is disposed in hollow body 22, and has a shape conforming to sidewall 34 in a region thereof about window 36.

Shutter member 24 is located in a track 37 in the hollow body. Track 37 takes the form of a shallow depression or recess with longitudinal edges or shoulders 39 serving as guides for the sliding shutter member 24. A transverse edge or shoulder 41 serves as an abutment to continued distal motion of shutter member 24 during an insertion stroke thereof. Shutter member 24 may be locked into track 37, for example, by grooves (not illustrated) in longitudinal edges or shoulders 39.

Hollow body 22 generally has a longitudinal axis 38, and sidewall 34 is formed with a bulging portion or protrusion 40 located on one side of the axis and extending from proximal end 32 of the hollow anoscope body partially along a length of sidewall 34 towards distal end 28. Window 36 is located in bulging portion 40, and shutter member 24 is slidable along and in engagement with bulging portion 40. Shutter member 24 and bulging portion 40 may be cooperatively formed so that the bulging portion serves as a track that slidably retains the shutter member. Window 36 may generally take any shape suitable for the admission of protruding hemorrhoidal tissues HT. Rectangular and circular are possible shapes.

Anoscope 20 is made of a metal or metal alloy material. To eliminate reflections from internal surfaces that interfere with proper visualization of anal tissues, anoscope 20 is provided along an inner surface 52, along lumen or channel 26, with a coating or layer 54 of a non-reflective or light-absorbing composition, such as a black polymeric paint. Preferably, the non-reflective coating 54 is applied along the entire inner surface 52 of the anoscope. Shutter member 24 is likewise provided along an inner surface with a coating or layer 56 of the non-reflective paint. Coating 54 and 56 may be provided by a spraying or atomizing technique.

Sidewall 34 and/or distal end 28 are optionally provided with one or more transparent polymeric windows 58, 60 to facilitate disposition of the anoscope during a hemorrhoidal treatment procedure. Those windows 58, 60 may be provided with a transparent anti-glare coating.

Hollow body 22 of anoscope 20 has a rim 42 surrounding opening 30 at proximal end 32. Hollow body 22 is provided along rim 42 with a flange 44 serving as a stop for preventing anoscope 20 from slipping entirely into the anal canal AC. Hollow body 22 is further provided along rim 42 with a cutout 46 disposed on a side of axis 38 opposite bulging portion 40. Cutout 46 facilitates manipulation of any instrument that is inserted into anoscope 20 for operating on hemorrhoidal tissues. In addition, cutout 46 facilitates observation of window 36 and of hemorrhoidal tissues HT protruding into longitudinal channel 26 through window 36.

In some applications, window 36 may extend in a proximal direction all the way to flange 44. In any case, window 36 is large enough for the admission of hemorrhoids into channel or lumen 26 of anoscope 20. The placement of window 36 in bulging portion or protrusion 40 enables the formation of window 36 with suitably large dimensions.

In a method for the treatment of hemorrhoids utilizing anoscope 20, anoscope 20 with shutter member 24 closing window 36 is inserted through an anal port member 89 (FIGS. 2A, 2B) into anal canal AC and is manipulated so that hemorrhoidal tissues HT are disposed adjacent to window 36. This procedure may involve longitudinally shifting and/or rotating the anoscope 20 inside the anal canal AC until the anoscope is in the desired position relative to the hemorrhoidal tissues HT.

Upon an appropriate positioning of anoscope 20, shutter member 24 is grasped at an external flange or finger grip 90 and pulled in a proximal direction, as indicated by an arrow 92 in FIG. 2B. This action uncovers window 36 and enables hemorrhoidal tissues HT to protrude through the window into channel 26 of anoscope 20. Subsequently, a distal end portion of a hemorrhoid treatment device (not shown) is inserted into anoscope 20. The hemorrhoidal tissues HT distal to an occluded neck region may be transected with a scalpel or allowed to is chemically regress or self amputate. Self-amputation occurs within a few days of the occlusion procedure. Ischemic regression takes place within several weeks. Ischemic regression and self-amputation are the result of occlusion of bloods vessels in the neck or base region.

Generally, the manipulating of anoscope 20 to align window 36 with hemorrhoidal tissues is performed after the inserting of anoscope 20 into the anal canal. A hemorrhoid treatment device is inserted into anoscope 20 after the inserting of anoscope 20 into the anal canal AC, after the manipulating of anoscope 20 to align window 36 with hemorrhoidal tissues HT, and after the protruding of the hemorrhoidal tissues HT through window 36.

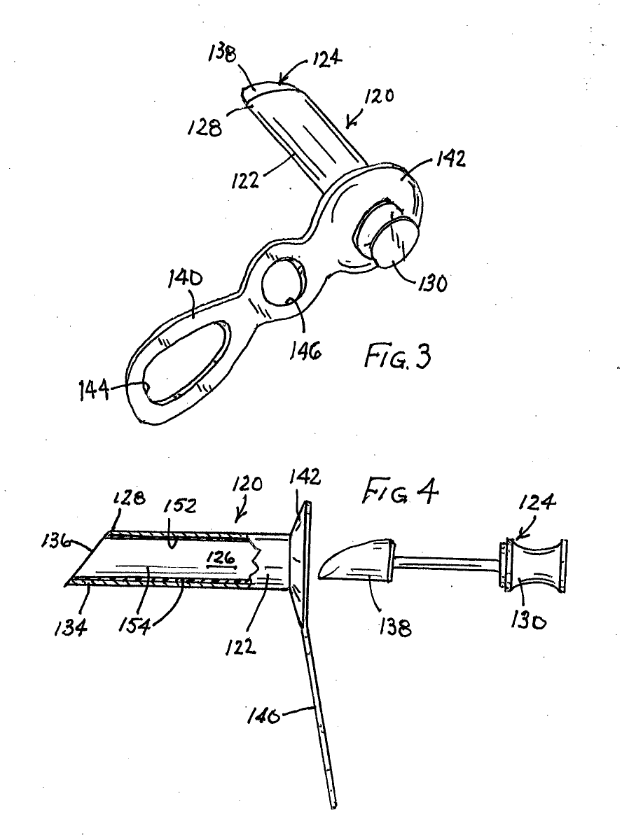

As illustrated in FIG. 3, another anoscope assembly 120 for hemorrhoidal surgery comprises a hollow anoscope body 122 and an obturator or insert member 124. Hollow body 122 defines a longitudinal channel or lumen 126 that has an opening or mouth 136 at a distal end 128 and formed with an opening (not visible) at a proximal end 132. The proximal opening enables visual inspection of a surgical site and the insertion of instrumentation.

Obturator 124 is movably mounted to hollow body 122 to cover or block distal end opening 136 and facilitate insertion and positioning of anoscope 120 in an anal canal. Obturator 124 is removable from channel or lumen 126 to permit access to hemorrhoidal tissues via distal end opening 136. More specifically, obturator 124 is slidably mounted to hollow body 122, is disposed in hollow body 122, and has a head 138 with a sidewall or outer surface (not separately designated) conforming to a sidewall 134 of anoscope 120. Obturator 124 has a finger grip 130 graspable by the user to alternately insert the obturator into channel 126 of anoscope 120 or to remove the obturator from the anoscope upon positioning thereof in an anal (or vaginal) canal.

Anoscope 122 includes a hand grip 140 extending from a shallow bowl-shaped flange 142 at a proximal end of the instrument. Hand grip 140 is formed with apertures 144 and 146 for finger insertion to facilitate handling.

Anoscope 120 is made of a metal or metal alloy material. To eliminate reflections from internal surfaces that interfere with proper visualization of anal tissues, anoscope 120 is provided along an inner surface 152, along lumen or channel 126, with a coating or layer 154 of a non-reflective or light-absorbing composition, such as a black polymeric paint. Preferably, the non-reflective coating 154 is applied along the entire inner surface 152 of the anoscope. Coating 154 may be provided by a spraying or atomizing technique.

Although the invention has been described in terms of particular embodiments and applications, one of ordinary skill in the art, in light of this teaching, can generate additional embodiments and modifications without departing from the spirit of or exceeding the scope of the claimed invention. In particular, the present invention applies to all manner of anoscopes. Accordingly, it is to be understood that the drawings and descriptions herein are proffered by way of example to facilitate comprehension of the invention and should not be construed to limit the scope thereof.

What is claimed is:

1. An anoscope comprising a hollow body defining a longitudinal channel, said hollow body being at least partially open at a proximal end, said hollow body being provided on an inner surface, facing said longitudinal channel, with a coating or layer of a non-reflective or light-absorbing material.

2. The anoscope defined in claim 1 wherein said coating or layer is made of a black material

3. The anoscope defined in claim 2 wherein said coating or layer is made of a black polymeric material.

4. The anoscope defined in claim 1 wherein said coating or layer is coextensive with said channel.

5. The anoscope defined in claim 1 wherein said coating or layer is applied to said sidewall.

6. The anoscope defined in claim 1, further comprising an insert movably mounted to said hollow body to cover an opening in said hollow body during a positioning of the anoscope in an anal canal, said insert being removable from said channel to permit hemorrhoidal tissues to protrude into said channel.
